(19) United States
(12) Patent Application Publication
(10) Pub. No.: US 2024/0378352 A1
(43) Pub. Date: Nov. 14, 2024

(54) A SYSTEM AND METHOD FOR MANUFACTURING HYBRID THERMOPLASTIC COMPOSITE MATERIAL

(71) Applicants: KARADENIZ TEKNIK UNIVERSITESI TEKNOLOJI TRANSFERI UYGULAMA VE ARASTIRMA MERKEZI, Ortahisar, Trabzon (TR); KAHRAMANMARAS SÜTÇÜ IMAM ÜNIVERSITESI, Kahramanmaras (TR)

(72) Inventors: Ayfer DÖNMEZ ÇAVDAR, Trabzon (TR); Tugrul ÇAVDAR, Trabzon (TR); Büsra AVCI, Kahramanmaras (TR); Fatih MENGELOGLU, Kahramanmaras (TR); Sevda BORAN TORUN, Trabzon (TR); Ercüment ÖZTÜRK, Trabzon (TR)

(21) Appl. No.: 18/557,518

(22) PCT Filed: Oct. 5, 2022
(86) PCT No.: PCT/TR2022/051092

§ 371 Date: Oct. 26, 2023

(30) Foreign Application Priority Data
Oct. 6, 2021 (TR) 2021/015560

(51) Int. Cl. G06F 30/27; G06F 113/26; G06F 119/18

(52) U.S. Cl. CPC G06F 30/27; G06F 2113/26; G06F 2119/18

(57) ABSTRACT

Disclosed is a system and method that makes it possible to produce hybrid thermoplastic composite materials at lower cost and in a shorter time with artificial intelligence optimization technique. The main objective of the system is the optimization and production of multi-component single-matrix hybrid composite materials with the aid of Particle Swarm Optimization (PSO). The particular objective of the system is to provide a product that has the potential for use for the automotive sector. The system is not designed on a single automotive part. However, with the achievement of the system targets, it creates the infrastructure for the development of special prototypes for the automotive sector.

### TECHNICAL FIELD

This invention relates to a system and method that provides for manufacturing hybrid thermoplastic composite materials at a lower cost and in a shorter time with artificial intelligence optimization technique.

### PRIOR ART

Many branches of industry, especially the automotive, space and defense industries, are always careful to use the most advanced level of the state-of-the-art technology and are increasing their desire to benefit from composite materials. The fact that composite materials, which are used as an alternative to other iron, aluminum, steel and similar materials in these areas, retain the potential to develop products with high resistance properties as well as the advantage of lightness by using low-production cost, renewable and environmentally friendly materials has attracted great attention in the sector and formed the basis of many studies.

The use of plastics and its derivatives in the automotive sector is increasing rapidly; this is due to technological developments aimed at increasing the efficiency and reducing fuel consumption in vehicles and inducing environmental awareness (Demirci, 2012).

However, in addition to these advantages, plastic materials also have disadvantages such as having soft surfaces, visco-elastic structure, low elasticity modulus values and low thermal stability. Due to these disadvantages, it is not possible to use polymers in pure (additive-free) form in areas of use where the above properties are expected. Composite materials using different additives and/or filling materials are preferred for such areas of use. The choice of filling material in composites and the homogeneous distribution of the filling material in the polymer are of great importance for obtaining the required properties. Although composite materials are mostly used to reduce the weight of automotives, improve their visual appearance and increase their crash performance (energy absorption), developments in the field of polymer composites (new materials, methods, etc.) have led to the preference of composites in other automotive parts where composites were not used before.

Increasing environmental pressures and the rapid increase in demand for resources in recent years have underlined the need to use resources more efficiently. This process has led to the development of a new term called “environmentally friendly”. One of the most important purposes of the use of environmentally friendly materials is undoubtedly to minimize waste generation while maximizing the efficiency of raw material use (Trost, 2002). The supply of raw materials and the fact that natural resources have come to the point of depletion is an important problem throughout the world. Composite panel production has been seen as an alternate method to the solution of this problem and has become an important field of research and application. Until recent years, single matrix and single reinforcement (additive) material were mostly used in composite products. However, the proliferation in the properties required by the sectors where composite material is used and environmental factors have made it more advantageous to produce “hybrid composites” with multi-component additives/reinforcing agents instead of a single matrix and a single reinforcing element in new product designs. However, in the hybrid composite product development process, the experimental study phases cause a great cost and therefore adversely affect the composite production financially. The use of artificial intelligence can provide significant convenience for industrial and research organizations that carry out R&D activities and that aim to produce new materials. Experiments can be moved to digital media with simulation methods; the number of these experiments can be reduced by using optimization techniques, and serious gains can be achieved in terms of cost, time and labor. It will be a serious advantage to benefit from artificial intelligence optimization techniques, especially in the development process of multi-component hybrid composite materials. Artificial intelligence technology is used in almost every field of use today. When we look at the literature, we see that artificial intelligence optimization algorithms are used in the field of composite production. Examples include the use of Gene Expression Programming (GEP) in polymer-carbon nanotube composite manufacturing modeling (Sabouhi et al. 2015), the use of Artificial Neural Networks (ANN) to predict the wear properties of polymer matrix composites (Jiang, 2007), the use of ANNs to make design optimizations, predict durability and other properties of composite materials (Zhang and Friedrich 2003). Furthermore, the design of reliable composite structures was made by using the Particle Swarm Optimization (PSO) algorithm together with Finite Element Analysis (Chen et al. 2013). In addition, the PSO algorithm was used to estimate the resistance capacity of connectors, especially in the field of civil engineering (Filiberto et al. 2010). By using artificial intelligence optimization algorithms in industry, experiments can be carried out in a digital environment and production costs can be reduced. Especially in cases where it is required to have the number of components increased, the number of experimental results increased and the inputs optimized depending on the outputs, artificial intelligence algorithms will provide significant gains in time and performance as well as cost.

In the presentation made at the 19th Annual Automotive Composites Conference and Exhibition (ACCE) of the Society of Plastic Engineers (SPE, Bethel, Conn., USA), organized by Ford Company, one of the locomotives of the automotive sector, which is held in Michigan in September 2019 and keeps the pulse of the automotive sector, five key areas were focused on, including artificial intelligence, which triggers change in the sector. These five key areas are sustainability, emerging materials, production of additives, advanced processes and design, and artificial intelligence.

When the studies conducted in the field of composites using PSO are examined, it is seen that one of the common points of the studies is PSO and another common point is that almost all of them are related to the processes or tests performed on the existing composites, not to the production of composites. This allows direct use of the PSO algorithm. However, a ready-made code block is used in the studies and this situation seriously restricts the ability to make intervention in the process. Because the process of determining raw material additive rates is more complex compared to the studies in the literature and requires an optimization (from test result values to determination of input parameters), using a ready-made code block proves to be insufficient. For this reason, in order to find raw material additive ratios, all coding studies should be recoded and they should be original so that they can be controlled. It is known that the use of PSO in the field of composite materials is quite common. The majority of studies on the use of PSO in composite material production in the literature relate to either physical or mechanical tests performed on composite materials (Vosoughi et al., 2016; Oliveira et al., 2017; Tao et al., 2017; Nazari and Javidrad, 2018; Khatir et al., 2019; Dadrasi et al., 2020; Keshtegar et al., 2020) or the shaping of a composite materials (Omkar et al., 2009; Chen et al., 2013; Hanafi et al., 2016). In addition to these, they presented a hybrid method with sensitivity method analysis and PSO techniques for damage assessment of laminated composite beams. This method provided a way to carry out the damage assessment process successfully (Vosoughi and Gerist, 2014). On the other hand, Chen and Wang (2019) used PSO in their studies to define the elastic component properties of epoxy composites and to determine other parameters when determining microstructural effects resulting from chemical reaction that cannot be easily detected from the manufacturing process and macroscopic behavior of composite materials. In recent years, the use of the PSO algorithm has become widespread rapidly in the field of composite materials and has involved in the solution of many different problems mentioned above.

In the patent application document numbered CN105608286B, which is in the known state of the art, the carbon fiber composite production method optimization is described for car bumper based on the particle swarm optimization algorithm. However, the said application document does not specifically mention the production of hybrid composites to be used under the hood and the determination of component additive ratios with PSO.

The patent application document numbered CN107563094A, which is in the known state of the art, mentions that the elastic performance of a three-dimensional woven carbon fiber composite material is optimized by using particle swarm optimization of material design variables such as estimation efficiency, the separation interval of fiber bundles and the number of fiber layers, and structural design variables. However, the said application document does not specifically mentions determination of component additive ratios with the PSO, the production of hybrid composites to be used under the hood, and the high mechanical and thermal resistance of the produced material.

The patent application document numbered CN111563347A, which is in the known state of the art, mentions a Plackett-Burman (PB) test design method to eliminate important factors affecting product quality from multiple parameters of design variables, and a Box-Behnken design (BBD) test design method to design a reasonable test scheme and an artificial intelligence-based particle swarm optimization (BP-AdaBoost-PSO) method developed as a parameter optimization method for the injection molding process using fiber-reinforced composite material in optimizing the injection molding process of the plastic part. However, the said application document does not specifically mentions determining the component additive ratios by PSO in hybrid composite production and the material having high thermal resistance.

Therefore, due to the inadequacy of the solutions to meet the needs described above, it is required to make an improvement in the relevant technical field.

### AIM OF THE INVENTION

The invention is inspired by the present situations and aims to solve the drawbacks mentioned above.

The aim of this invention is to optimize and produce multi-component single-matrix hybrid composite materials with the aid of Particle Swarm Optimization (PSO).

Another aim of this invention is to provide a product that will have the potential to be used for the automotive sector.

The structural and characteristic features of the invention and all its advantages will be understood more clearly by means of the figures presented below and the detailed description written by reference to these figures. For this reason, the evaluation should be made by considering these figures and the detailed description.

### DRAWINGS TO HELP UNDERSTAND THE INVENTION

FIG. 1 is a schematic representation of the system, which is the subject of the invention.

FIG. 2 is the flow diagram of the method, which is the subject of the.

### DESCRIPTION OF PART REFERENCES

1. System
2. Mixing module
3. Test module
4. Production module
1000. Method

### DETAILED DESCRIPTION OF THE INVENTION

The preferred embodiments of the system (1) and method (1000) that are the subject of the invention are explained in this detailed description only for the purpose of better understanding the subject.

This invention relates to a system (1) and method (1000) that makes it possible to produce hybrid thermoplastic composite materials at lower cost and in a shorter time with artificial intelligence optimization technique.

The main aim of the system (1) is the optimization and production of multi-component single-matrix hybrid composite materials with the aid of Particle Swarm Optimization (PSO). The particular objective of the system (1) is to provide a product that has the potential for use for the automotive sector. The system (1) is not designed on a single automotive part. However, with the achievement of the system (1) targets, it creates the infrastructure for the development of special prototypes for the automotive sector.

The criteria to need to be met in automotive parts are examined in the system (1). When we look at the place of use of polymeric materials and their proportions in automotive we see that the hybrid composite produced by the system (1) is a potential product for under-the-hood applications. The product produced in the system (1) has higher mechanical and thermal resistance than the pure polymer (polypropylene homopolymer).

Even though the use of (1) PSO in the system has a common point with other studies in the literature, the system differs from other studies in that it is used in the first stage of composite material production (determining the additive ratios of the components before producing the material), that the algorithm is fully coded in accordance with the purpose of the project, and that no ready-made code block or package program is used. PSO steps are dominant in the entire process in the system (1) from the first step of the composite material production stage until final product is produced. Because, properties and usage amounts of each of the materials included in the mixture are criteria for the algorithm. Therefore, the algorithm and the production process are complementary to each other in the system (1). The difference of the system (1) from other studies is that the PSO is used in the most basic production, that is, in the direct production of the composite material and in all the processes to be performed on the produced composite material.

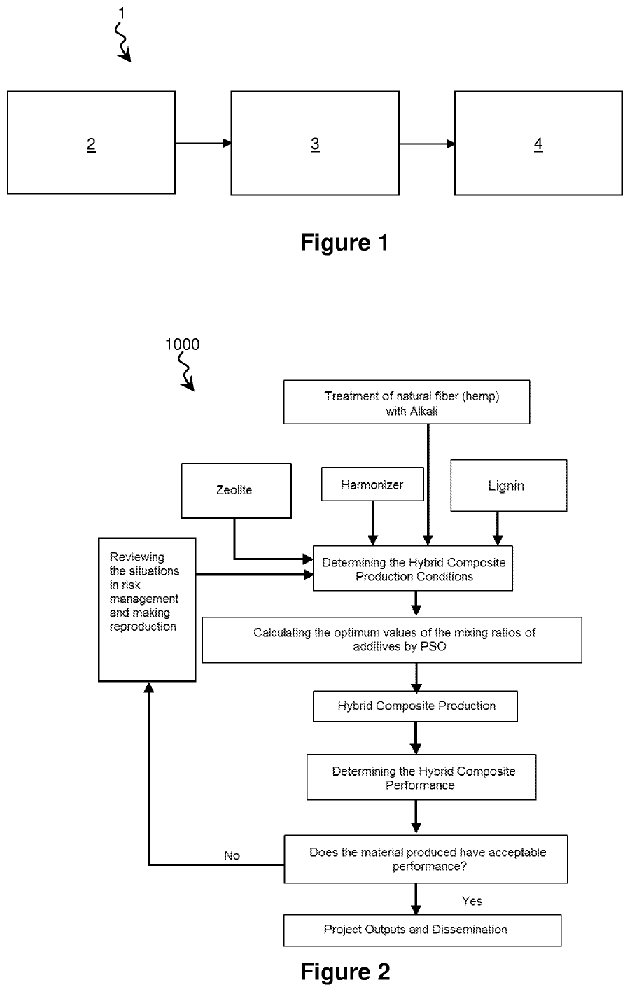

The system (1) illustrated in FIG. 1, which is the subject of the invention, comprises following modules.
Mixing module (2), which enables the optimization of raw material additive ratios through the Particle Swarm Optimization (PSO) algorithm, thus achieving the ideal ratio of mixing for the components of the composite material before the production of the hybrid thermoplastic composite material,
Test module (3), which reduces the number of tests by performing simulation studies through the Particle Swarm Optimization (PSO) algorithm and in turn, reduces the cost, and
Production module, which uses the Particle Swarm Optimization (PSO) algorithm and produces high quality hybrid thermoplastic composite material that meets the necessary criteria for under-the-hood and similar applications by means of the artificial intelligence techniques it uses (4).

The system (1) makes contribution to the environment and energy on a sustainable basis. The use of petroleum-derived thermoplastics is reduced in the system (1) and hybrid composite optimization is provided by the use of more environmentally friendly and sustainable resources. By means of the system (1), contribution is made to the transformation of the lignin, which has the potential to be obtained from paper mill wastes worldwide, into products with high added value and also contribution is made to world waste management. In this way, contribution is made to the improvement of people's quality of life, welfare and education level.

The system (1) provides contribution to technological and academic innovations in many areas such as new R&D decisions, national/international R&D collaborations, change in the number and quality of researchers and contribution to university-industry collaborations. By means of the artificial intelligence methods used in the system (1), a structure is presented that will set an example for interdisciplinary new studies in the field of composite material production.

The system has (1) economic implications. The hybrid composite material with optimum mixing ratios produced in the system (1) has the potential to be applied in the automotive sector. By means of the system (1), R&D costs of the hybrid composite material manufacturer and user sectors are reduced in the process of product development by PSO method by reducing the number of production and tests. The system (1) contributes to employment and increases competitiveness. With the increase in competitiveness, development is achieved in platforms such as export and import, the formation of new firms and the triggering of foreign capital investment.

The system (1) provides for the enhancement of security systems such as cyber security, energy security, border security, economic security and security of similar types. Considering that the level of development of a country is identified with the proportion of plastic used in the automotive sector in that country, using agricultural wastes that has no added value and the use of plastic in the automotive sector by means of the sustainable material developed by the system (1) help to achieve Turkey's eleventh development plan (Articles 359 and 382) and 2023 targets.

One of the most well-known examples of artificial intelligence algorithms is the particle swarm optimization (PSO) algorithm. PSO is a heuristic method and it is a stochastic optimization method inspired by the movements of flocks of birds and fish to find optimal results for nonlinear problems (Kennedy and Eberhart, 1995). In the PSO, the individual who searches to find the overall solution and each of which is the candidate solution is called a particle, and the community in which the particles are located is called a swarm. In the system (1), each particle represents an experiment to be performed in a simulated environment, and a swarm represents an experiment that is optimal from all test results. Each particle is affected by the best value it obtains and the best value range obtained by the flock; however, it tends to move randomly during the simulation. In other words, while each experiment is independent in itself, it produces a solution that converges to all possible results. The fitness function is used to understand how close an individual is to the solution

Even though the use of (1) PSO in the system has a common point with other studies in the literature, the system differs from other studies in that it is used in the first stage of composite material production (determining the additive ratios of the components before producing the material), that the algorithm is fully coded in accordance with the purpose of the project, and that no ready-made code block or package program is used. PSO steps are dominant in the entire process in the system (1) from the first step of the composite material production stage until final product is produced.

Because, properties and usage amounts of each of the materials included in the mixture are criteria for the algorithm. Therefore, the algorithm and the production process are complementary to each other. In summary, the difference of the system (1) from other studies is that the PSO is used in the most basic production, that is, in the direct production of the composite material and in all the processes to be performed on the produced composite material.

The flow algorithm of the method (1000), which is the subject of the invention, is illustrated in FIG. 2 and it comprises the following processing steps:
treatment of natural fiber (hemp) with alkali with the mixing module (2),
determining the hybrid composite production conditions by adding alkali-treated natural fiber (hemp) zeolite, harmonizer and lignin with the mixing module (2);
calculating the optimum values of the mixing ratios of additives by PSO with the test module (3),
producing the hybrid composites by means of the production module (4),
· determining the hybrid composite performance with the production module (4),
checking with the production module (4) whether the material produced has acceptable performance,
if the produced material does not have acceptable performance, reviewing the situations in risk management with the test module (3) and making reproduction with the production module (4), and
if the produced material has acceptable performance, sharing and disseminating the project outputs with the production module (4).

In the method (1000) used in the system (1,) polypropylene is used as a polymer matrix, which is mostly preferred in the automotive industry. The additives of the system (1) are planned as lignin, natural fiber, zeolite and polypropylene grafted with harmonizing agent-maleic anhydride. Hemp stalks are preferred as natural fiber. Increasing the inter-surface adhesion force between the polymer matrix by treating the natural fibers with alkali was previously pre-studied with different natural fibers and it was found that there was an improvement of 10-15% in mechanical properties of the produced composite samples. It was taken into account that these substances should have similar characteristics with each other in order to be homogeneously distributed in the matrix in the selection of additives in the system (1). As a result of experimental studies, it has been observed that hemp stalks treated with alkali with lignin, which is alkaline, show a more homogeneous distribution in polymer than hemp stalks that are not treated with lignin.

The mixing module (2) adds the NaOH solution to the alkali-treated hemp fibers. The mixing module (2) then adds lignin and zeolite to this solution and then dries the mixture. The mixing module (2) mixes it by adding a harmonizing and thermoplastic polymer to the dried mixture. The mixing module (2) performs extrusion, crushing and injection on the final mixture.

The mixing module (2) determines the operating conditions of the machines required for production and the places of use in production and determines the extrusion and injection temperatures and auger rotation rates needed for production. The mixing module (2) first puts the hemp stalks through a preliminary cleaning process. The mixing module (2) then grinds the hemp stalks at Willey's mill to homogeneous particle sizes and, if necessary, performs sieving.

The mixing module (2) subjects the hemp stalks to 24-hour drying process at 103° C. in the in laboratory type drying oven before the alkaline treatment process and bringing it to a full dry weight, treats the ground hemp stalks with 5% NaOH for 24 hours as the effect of alkaline treatment will be examined, thereby removing unwanted soluble hemicellulose, pectin, extracts from the hemp stalks, washes the fibers with distilled water after alkaline treatment to remove excess NaOH, and dries the fibers at 60° C. for 24 hours.

The test module (3) creates a simulation environment by coding it using the Particle Swarm Optimization (PSO) algorithm. Each composite material that is likely to be produced by simulation is considered a particle, the best particle is found, and this continues until a desired error rate or maximum number of iteration (process steps) is reached. In this manner, the result or results obtained as a result of the simulation run in the computer environment (since particles (composites) that are close to each other regarding quality will also be considered as an efficient result) and during the production of hybrid composites with thermoplastic matrix with lignin content, it is determined which raw material is required and in what proportion and it is checked whether the selected additives/filling materials provide acceptable properties. In addition, based on observation during the production phase, the surface evenness, fluidity and pressure increase of the material are checked during production.

The test module (3) initiates PSO with all possible particles, calculates the conformity value of the test results, updates the best of all composite products likely to be obtained locally and globally (pbest, gbest), updates the exchange and position of all particles, checks whether the termination criteria are met according to the maximum iteration or desired error rate, recalculates the conformity value of the test results if the termination criteria are not met and repeats the procedures and terminates the PSO algorithm if the termination criteria are met.

The test module (3) finds the best of all composite products (particles) that are likely to be obtained with the PSO algorithm. Then test module (3) runs the simulation in reverse in order to produce the best composite and finds the mixture ratios of the best products. The test module (3) updates these ratios by looking at the standard deviation, variance and average values in all productions. Thus test module (3) creates a new mixture recipe with the new determined ratios and performs new productions based on these recipes. The test module (3) makes comparisons with previous products by performing tests on new productions and determines the performance rate in these comparisons. The test module (3) provides significant advantages both in terms of obtaining a higher quality product and eliminating the cost of numerous tests to obtain a higher quality product.

The production module (4) makes it possible to make new productions and produce products with the recipes obtained as a result of simulation studies. Tests are performed on these obtained products in the laboratory environment and the conformity values of the test results are compared. The results obtained are proof of system's (1) performance and accuracy. The accuracy and usability of the system has been proven as a result of simulation works, recipes obtained from these works and productions made with these recipes.

The production module (4) is configured to perform the following processes:
obtaining production recipes with PSO simulations,
making productions in accordance with prescriptions,
subjecting productions to tests,
examination of the conformity values of the test results,
if the test results are not acceptable, returning to the process of obtaining production recipes with PSO simulations, and
if the test results are acceptable, making the production of the final product.

1. A system that makes it possible to produce hybrid thermoplastic composite materials at lower cost and in a shorter time with artificial intelligence optimization technique, the system comprising:
a mixing module which enables the optimization of raw material additive ratios through the Particle Swarm Optimization (PSO) algorithm, thus achieving an ideal ratio of mixing for the components of the composite material before the production of the hybrid thermoplastic composite material;
a test module which reduces the number of tests by performing simulation studies through the Particle Swarm Optimization (PSO) algorithm and in turn, reduces the cost; and
a production module which uses the Particle Swarm Optimization (PSO) algorithm and produces high quality hybrid thermoplastic composite material that meets the necessary criteria for under-the-hood and similar applications by means of the artificial intelligence techniques it uses.

2. The system according to claim 1, comprising a mixing module that adds NaOH solution to an alkali hemp treatment, then adds lignin and zeolite to this solution, and then dries the mixture, mixes it by adding harmonizing and thermoplastic polymer to the dried mixture, and performs extrusion, crushing and injection on the finished mixture.

3. The system according to claim 1, wherein the mixing module determines the operating conditions of the machines required for production and the places of use in production, determines the extrusion and injection temperatures and auger cycle rates needed for production, first putting the hemp stalks through a preliminary cleaning process, then grinding the hemp stalks in the Willey mill to homogeneous particle sizes and, if necessary, performing the sieving process, subjecting the hemp stalks to 24-hour drying process at 103° C. in the in laboratory type drying oven before the alkaline treatment process and bringing it to a full dry weight, treating the ground hemp stalks with 5% NaOH for 24 hours as the effect of alkaline treatment will be examined, thereby removing unwanted soluble hemicellulose, pectin, extracts from the hemp stalks, washing the fibers with distilled water after alkaline treatment to remove excess NaOH, and drying the fibers at 60° C. for 24 hours.

4. The system according to claim 1, wherein the test module initiates PSO with all possible particles, calculates the conformity value of the test results, updates the best of all composite products likely to be obtained locally and globally (pbest, gbest), updates the exchange and position of all particles, checks whether the termination criteria are met according to the maximum iteration or desired error rate, recalculates the conformity value of the test results if the termination criteria are not met and repeats the procedures and terminates the PSO algorithm if the termination criteria are met.

5. The system according to claim 1, wherein the test module finds the best of all composite products (particles) that are likely to be obtained with the PSO algorithm, then runs the simulation in reverse in order to produce the best composite and finds the mixture ratios of the best products, updates these ratios by looking at the standard deviation, variance and average values in all productions, thus creates a new mixture recipe with the new determined ratios and performs new productions based on these recipes, makes comparisons with previous products by performing tests on new productions and determines the performance rate in these comparisons.

6. The system according to claim 1, wherein the production module is configured to perform the following processes:
obtaining production recipes with PSO simulations,
making productions in accordance with prescriptions,
subjecting productions to tests,
examination of the conformity values of the test results,
if the test results are not acceptable, returning to the process of obtaining production recipes with PSO simulations, and
if the test results are acceptable, making the production of the final product.

7. A method that makes it possible to produce hybrid thermoplastic composite materials at lower cost and in a shorter time with artificial intelligence optimization technique, comprising the following process steps:
treatment of natural fiber (hemp) with alkali with a mixing module;
determining the hybrid composite production conditions by adding alkali-treated natural fiber (hemp) zeolite, harmonizer and lignin with the mixing module;
calculating the optimum values of the mixing ratios of additives by PSO with the test module;
producing the hybrid composites by means of the production module;
determining the hybrid composite performance with the production module;
checking with the production module whether the material produced has acceptable performance;
if the produced material does not have acceptable performance, reviewing the situations in risk management with the test module and making reproduction with the production module; and
if the produced material has acceptable performance, sharing and disseminating the project outputs with the production module.
